(19) United States
(12) Patent Application Publication
(10) Pub. No.: US 2007/0032685 A1
(43) Pub. Date: Feb. 8, 2007

(54) Process for the preparation of 1-(2,2,6-trimethylcyclohexyl)-hexan-3-ol

(75) Inventor: Dietmar Schatkowski, Stadtoldendorf (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Symrise GmbH & Co., KG, Holzminden (DE)

(21) Appl. No.: 11/498,257

(22) Filed: Aug. 3, 2006

(30) Foreign Application Priority Data
Aug. 4, 2005 (DE) 10 2005 036 672.4

(51) Int. Cl. C07C 35/08

(52) U.S. Cl. 568/822

(57) ABSTRACT

A process for the preparation of 1-(2,2,6-trimethylcyclohexyl)-hexan-3-ol of the formula A 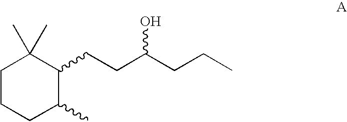 having a content of at least 30 wt. % of the corresponding trans isomer, based on the total amount of 1-(2,2,6-trimethylcyclohexyl)-hexan-3-ol of the formula A, with the following step:
Catalytic hydrogenation of a compound of the formula D 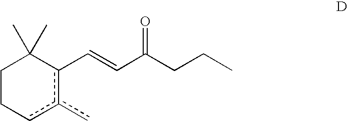 in which the broken lines represent a single double bond which is located in one of the three positions drawn in, in the presence of a rhodium catalyst to give 1-(2,2,6-trimethylcyclohexyl)-hexan-3-ol of the formula A.

The present invention relates to a process for the preparation of 1-(2,2,6-trimethylcyclohexyl)-hexan-3-ol of the formula A 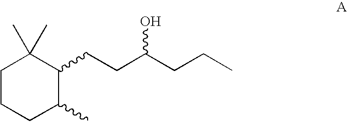
having a content of at least 30% by weight of the corresponding trans isomer of the formula B, based on the total amount of 1-(2,2,6-trimethylcyclohexyl)-hexan-3-ol of the formula A. In addition to the trans isomer, the cis isomer of the formula C is present here.

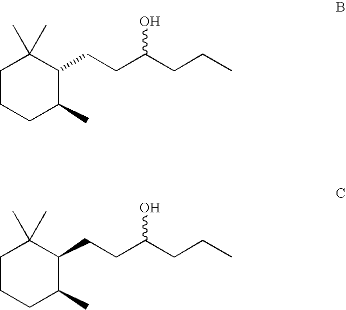

The invention also relates to a process for the preparation of a corresponding perfume composition.

1-(2,2,6-Trimethylcyclohexyl)-hexan-3-ol of the formula A (including the isomers of the formulae B and C) is a compound which has found a ready use in the preparation of perfume compositions because of its characteristic woody/ambered odour and the good fixing properties and actions, cf. DE 24 55 761 and DE 28 07 584.

The compound of the formula A can be prepared e.g. via the following reaction route

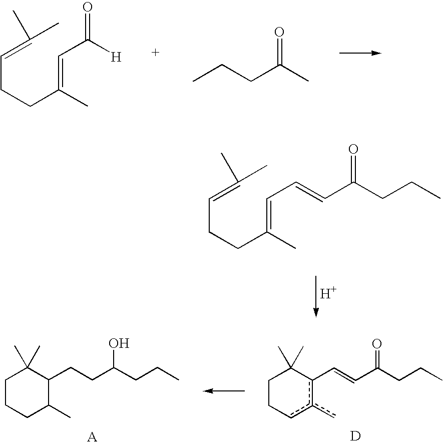

In this context, the broken lines drawn in formula D represent a single double bond which can be located in one of the three positions drawn in. Using the conventional nomenclature for ionones, this is therefore a double bond in the α,β or y positions (compare Rompp-Lexikon “Naturstoffe” [Römpp Dictionary “Natural Substances”], Thieme Verlag, 1997, page 334-335)

The compounds of the formula D can be prepared here by reactions with which the person skilled in the art is familiar, such as e.g. by condensation of citral with 2-pentanone and subsequent acid-catalysed cyclization; compare Helv. Chim. Acta 1948, 31, 417.

DE 24 55 761 discloses that in the hydrogenation of e.g. methylionones in the presence of Raney nickel as the sole catalyst, only 12% of the theoretically possible amount of hydrogen is taken up. According to DE 24 55 761, a hydrogenation in the presence of Raney nickel alone does not lead to reduction of the carbonyl group, this taking place only if Raney nickel and copper chromite are present simultaneously.

EP 1 400 503 discloses that, in particular, if a base is added and certain temperature and pressure ranges are adhered to, complete hydrogenation with Raney nickel is successful even in the absence of copper chromite, at least 15% of the sensorially more valuable trans isomers of the formula B being formed.

DE 100 62 771 discloses two alternative process variants. According to a first process variant, 1-(2,2,6-trimethyl-1- or -2-cyclohexen-1-yl)-1-alken-3-ones are reduced to the corresponding 1-(2,2,6-trimethylcyclohexyl)-3-alkanols having a content of 20% of trans isomers using ruthenium catalysts. The reaction here is preferably carried out under a hydrogen pressure in the range of from 10 to 20 bar at a temperature of from 130° C. to 150° C. However, the reaction time of 60 hours, which is very slow for an industrial reaction, is a disadvantage of this first process variant according to DE 100 62 771.

According to the second process variant disclosed in DE 100 62 771, 1-(2,2,6-trimethylcyclohex2-enyl)alk-1-en-3-ols are first prepared by a technically involved reduction of 1-(2,2,6-trimethyl-1- or -2-cyclohexen-1-yl)-1-alken-3-ones with sodium borohydride, and these are then hydrogenated in the presence of hydrogenation catalysts based on elements of sub-groups Ib, VIb and VIIIb. Rhodium is mentioned, inter alia, as a hydrogenation catalyst which can be employed, and the hydrogenation is particularly preferably carried out under a hydrogen pressure in the range of from 5 to 15 bar at a temperature in the range of from 20° C. to 50° C., a content of trans isomers in the range of between 30% and 40% preferably being obtained.

Helv. Chim. Acta 1999, 82, 1762-1773 describes the hydrogenation of 1-(2,6,6-trimethylcyclohex-2-enyl)-hex-1-en-3-ols using Pd/C or PtO₂. At room temperature, 1-(2,2,6-trimethylcyclohexyl)-hexan-3-ols of the formula A were obtained in the presence of 30 wt. % of Pd/C (10% on active charcoal) in acetic acid under a pressure of 3 bar, the ratio of trans isomers of the formula B to cis isomers of the formula C being 40:60.

According to P. N. Rylander [Catalytic Hydrogenation in Organic Synthesis, Academic Press 1979, ISBN 0-12-605355-3], the hydrogenation catalysts differ within a wide range in respect of activity and selectivity. The elements of group VIIIb are regarded generally as catalysts for the low pressure range. Thus, for example, reference is made in Adv. Catal., 9, 773, (1957) to the adequate activity at low temperatures and pressures for carrying out hydrogenations.

In J. Amer. Chem. Soc. 1962, 84, 3132-3136, the catalytic hydrogenation of 4-tert-butyl-1-methylenecyclohexane was investigated. If the hydrogenation of 4-tert-butyl-1-methylenecyclohexane is carried out with palladium under low pressure, more trans compound is obtained compared with results found with platinum oxide. When rhodium or ruthenium were employed as hydrogenation catalysts, in each case significantly more or the corresponding cis compound was obtained compared with the process designs employing platinum oxide or palladium.

These and other works show that after hydrogenations in the presence of hydrogenation catalysts with elements of sub-group VIIIb, product compositions show significant differences depending on the reaction conditions, and general predictions of the stereochemical product compositions to be expected cannot be made.

Further publications describe the preparation of compounds of the formula A with the highest possible content of trans isomer of the formula B. Reference may be made in this respect in particular to EP 0 118 817 and EP 0 456 932. However, the processes described therein are not very suitable for an industrial scale either because of the use of reagents which are difficult to handle, such as e.g. lithium aluminium hydride or sodium borohydride, or because of their many stages.

As already disclosed in EP 0 118 817 A1, the trans compound of the formula B and the isomeric cis compound of the formula C are distinguished in that they have different odour profiles. EP 0 118 817 A1 describes isomer mixtures having a content of trans-1-(2,6,6-trimethylcyclohexyl)-hexan-3-ols of the formula B of at least 60% and a content of cis 1-(2,6,6-trimethylcyclohexyl)-hexan-3-ols of the formula C of not more than 40%.

In “Riechstoffe und Geruchssinn [Odoriferous Substances and Olfactory Sense]” (Springer Verlag Heidelberg 1990; ISBN 3-540-52560), the isomeric compounds are described in terms of odour as follows: the cis-1-(2,6,6-trimethylcyclohexyl)hexan-3-ols of the formula C smell woody-ambered, coupled with a sweaty nuance. The trans-1-(2,6,6-trimethylcyclohexyl)-hexan-3-ols of the formula B have a decidedly animal and ambergris character, the animal note already being attributed clearly unpleasant, faecal, stercoral odour impressions. However, sweaty (cis) or faecal (trans)-odour impressions are undesirable in the perfuming of commercial products.

EP 0 118 809 A2 describes mixtures of 1-(2,6,6-trimethylcyclohexyl)-hexan-3-ols comprising at least 50 wt. % of the trans isomers B and not more than 50 wt. % of the cis isomers C as being suitable for most fields of perfumery.

Reference may furthermore be made to the following documents: Houben-Weyl, Methoden der org. Chemie [Methods of Org. Chemistry], volume IV/1c, page 192-193 and E. Breitner, E. Roginski & P. N. Rylander, J. Org. Chem. 24, 1855 (1959). Changes in the isomer composition towards a lower trans content than 50% wt. % lead to significantly more woody odour impressions with a suggestion of vetiver oil. The product marketed under the product name Timberol, having a content of trans isomer of the formula B of 10-12%, is even described as “cedarwood-like”. This odour impression makes the product seem rather less valuable perfumistically, since cedarwood oil is available in large quantities and is employed in large quantities for perfuming inexpensive commercial products.

It was an object of the present invention to provide a process for the preparation of 1-(2,6,6-trimethylcyclohexyl)-hexan-3-ols of the formula A wherein an isomer mixture which has neither the negative faecal odour impressions of the pure trans isomers of the formula B nor the sweaty odour impressions of the cis isomers of the formula C to a troublesome extent should preferably be prepared.

Preferably, the preparation process to be provided should start from easily accessible and inexpensive educts and provide the desired product in only one reaction step and with a comparatively short reaction time, so that the process is also suitable for the industrial scale.

Finally, the process to be provided should also render possible a high conversion of the educts to be employed, in order to be able to achieve comparatively pure product mixtures in this manner.

The stated object is achieved according to the invention by a process for the preparation of 1-(2,2,6-trimethylcyclohexyl)-hexan-3-ol of the formula A 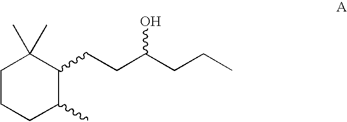
having a content of at least 30 wt. % of the corresponding trans isomer, based on the total amount of 1-(2,2,6-trimethylcyclohexyl)-hexan-3-ol of the formula A, with the following step:

Catalytic hydrogenation of a compound of the formula D 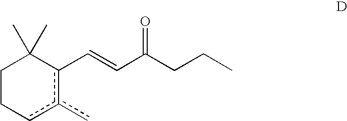
in which the broken lines represent a single double bond which is located in one of the three positions drawn in, in the presence of a rhodium catalyst to give 1-(2,2,6-trimethylcyclohexyl)-hexan-3-ol of the formula A.

Formula D includes the following compounds: 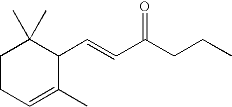
(1E)-1-(2,6,6-trimethylcyclohex-2-en-1-yl)hex-1-en-3-one 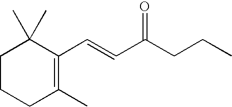
(1E)-1-(2,6,6-trimethylcyclohex-1-en-1-yl)hex-1-en-3-one 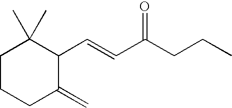
(1E)-1-(2,2-dimethyl-6-methylenecyclohexyl)hex-1-en-3-one

The process according to the invention renders possible, in a simple manner, the preparation of 1-(2,6,6-trimethylcyclohexyl)-hexan-3-ols of the formula A, wherein the content of trans isomer of the formula B in the reaction product is preferably even in the range of from 40 wt. % to 50 wt. %, based on the total amount of 1-(2,6,6-trimethylcyclohexyl)-hexan-3-ol of the formula A, depending on the choice of reaction conditions. The content of the cis isomer is accordingly preferably in the range of from 60 wt. % to 50% wt. %. The corresponding trans/cis isomer ratio can be established according to the invention (a) without an additional (expensive) distillation step and (b) without prior reduction of the carbonyl group of the compound of the formula D employed as an educt (using reagents which are to be employed according to the prior art, such as e.g. lithium aluminium hydride or sodium borohydride). This clearly differentiates the process according to the invention from the process variants according to DE 100 62 771 A1.

Preferably, in the process according to the invention the hydrogenation is ended only after conversion of at least 98% of the compound of the formula D.

In the process according to the invention, the hydrogenation is preferably carried out in a hydrogen pressure range of 1-200 bar. Establishing a hydrogen pressure in a preferred range of 80-200 bar and in a particularly preferred range of 80-150 bar renders it possible, in a particularly simple manner, to achieve a content of from 40 wt. % to 50 wt. % of the trans isomer in the isomer product mixture prepared, the percentage data here again relating to the total amount of 1-(2,6,6-trimethylcyclohexyl)-hexan-3-ol of the formula A prepared. A hydrogen pressure in the range of from above 100 bar to 200 bar is particularly preferred. The range of from above 100 bar to 150 bar is very particularly preferred. A further important difference from the known prior art is to be seen in particular in the choice of the preferred hydrogen pressure ranges (in combination with the choice of catalyst).

In the process according to the invention, the catalytic hydrogenation is preferably carried out at a temperature in the range of from 20° C. to 300° C., preferably at a temperature in the range of from 180° C. to 260° C. That stated above applies here in respect of the preferred hydrogen pressure ranges.

A rhodium catalyst is employed in the process according to the invention. Preferably, the rhodium is employed here in an amount of from 0.0001 to 10 wt. %, preferably in the range of from 0.01 to 0.5 wt. %, based on the amount of compound of the formula D employed.

The rhodium used as the catalyst is preferably applied to a support material. Support materials such as active charcoal, aluminium oxide or silicon oxide are preferred. The amount of rhodium catalyst on the support material is preferably in the range of from 5 to 10 wt. %, based on the total weight of support material and rhodium.

Particularly good results were achieved in our own investigation when rhodium catalysts were used on active charcoal or another support material, such as aluminium oxide, in an amount of from 0.0001 to 10 wt. %, preferably of from 0.01 to 0.5 wt. %, based on the amount of compound of the formula D employed. Higher amounts of catalyst here regularly lead to comparatively high contents of trans isomers (formula B) and render short reaction times possible (cf. Examples 1-3 below).

The process according to the invention can be carried out both continuously and discontinuously.

In the case of a discontinuous reaction procedure (batch), the rhodium catalyst is preferably employed as a powder.

Because of the short reaction times, the process according to the invention can also be carried out in fixed bed reactor, such as, for example, a trickle bed reactor, on a fixed catalyst bed. In the case of a continuous reaction procedure, in some cases it is advantageous to use the rhodium catalyst as shaped bodies or hollow bodies. Bodies in the shape of hollow strands, extrudates, pellets, extruded mouldings, spiral strands, saddles, rings, hollow beads, beads, hollow cylinders, cylinders, cubes, tablets, cones and the like can be employed here in particular.

In a process according to the invention, the step of catalytic hydrogenation can be carried out either in substance (i.e. solvent-free) or in solution. Suitable solvents for the educt compound of the formula D and the product compound of the formula A are substances such as alcohols (e.g. methanol, ethanol, isopropanol), esters (e.g. ethyl acetate) or hydrocarbons (e.g. hexane or cyclohexane) as well as mixtures thereof.

Starting from a compound of the formula D, the process according to the invention using rhodium catalysts leads to a reaction product of the formula A having a high content of trans isomers of the formula B, and in particular in a single reaction step without a prior reduction (hydrogenation) of the oxo group. The educt compounds of the formula D are readily accessible compounds, so that the process according to the invention can be carried out overall with only little technical outlay. The process according to the invention furthermore leads to a particularly complete conversion of the educt compounds of the formula D. A degree of conversion of >98% can be achieved surprisingly easily. The complete conversion leads finally to an improved quality of the products and facilitates purification thereof.

According to a preferred embodiment, in a process according to the invention

(a) the 1-(2,2,6-trimethylcyclohexyl)-hexan-3-ol of the formula A prepared is separated off

(i) from the rhodium catalyst and/or

(ii) from other constituents of the product mixture and/or

(b) an isomer of the 1-(2,2,6-trimethylcyclohexyl)-hexan-3-ol of the formula A prepared is separated off from the product mixture or concentrated.

The present invention also relates to a process for the preparation of a perfume composition, with the following steps:
Preparation of 1-(2,2,6-trimethylcyclohexyl)-hexan-3-ol by a process according to the invention (as described above).
Optionally isolation of the 1-(2,2,6-trimethylcyclohexyl)-hexan-3-ol from the product mixture and/or purification of the product mixture,
   and subsequently
mixing of a sensorially active amount of the 1-(2,2,6-trimethylcyclohexyl)-hexan-3-ol with one or more further odoriferous substances.

In this context, that stated above of course applies in respect of preferred embodiments in the preparation of the 1-(2,2,6-trimethylcyclohexyl)-hexan-3-ol.

In a perfume composition prepared according to the invention, the content of 1-(2,2,6-trimethylcyclohexyl)-hexan-3-ol of the formula A is preferably in the range of from 0.0001 to 50 wt. %, preferably in the range of from 0.01 to 20 wt. %, based on the total weight of the perfume composition. Preferably, the content here of trans isomers of the formula B is in the range of from 40 to 50 wt. %, based on the total weight of the trimethylcyclohexyl-hexan-3-ol of the formula A.

The invention is explained further in the following with the aid of examples. Unless stated otherwise, in the examples all the data relate to the weight. The abbreviation DPG stands for dipropylene glycol.

### EXAMPLE 1 (According to the Invention)

### Preparation of 1-(2,2,6-trimethylcyclohexyl)-hexan-3-ol

2,000 g of a mixture of 1-(2,6,6-trimethylcyclohex-1-en-1-yl- or -2-en-1-yl-)-hex-1-en-3-one (according to GC approx. 80%) were hydrogenated with 0.4 g of 10% rhodium on charcoal, corresponding to 0.02 wt. % of rhodium on active charcoal, in a stirred autoclave with a gassing stirrer under 100 bar and at a reaction temperature of 200° C.-220° C. for 2 hours. Heating up was carried out in the course of 45 minutes. After filtration and distillation, 1,560 g of completely hydrogenated product were obtained. The weight ratio of trans-1-(2,2,6-trimethylcyclohexyl)hexan-3-ol of the formula B to cis-1-(2,2,6-trimethylcyclohexyl)-hexan-3-ol of the formula C was 40:60.

### EXAMPLE 2 (According to the Invention)

### Preparation of 1-(2,2,6-trimethylcyclohexyl)-hexan-3-ol

2,000 g of a mixture of 1-(2,6,6-trimethylcyclohex-1-en or 2-en-1-yl)-hex-1-en-3-one (according to GC approx. 80%) were hydrogenated with 1 g of 10% rhodium on active charcoal, corresponding to 0.05 wt. % of rhodium on active charcoal, in a stirred autoclave with a gassing stirrer under 100 bar and at a temperature of from 180° C. to 260° C. for 1 hour. Heating up was carried out here in the course of 30 minutes. After filtration and distillation, completely hydrogenated product was obtained (M2; cf. Example 7). The weight ratio of trans-1-(2,2,6-trimethylcyclohexyl)-hexan-3-ol of the formula B to cis-1-(2,2,6-trimethylcyclohexyl)-hexan-3-ol of the formula C was 49:51.

### EXAMPLE 3 (According to the Invention)

### Preparation of 1-(2,2,6-trimethylcyclohexyl)-hexan-3-ol

2,000 g of a mixture of 1-(2,6,6-trimethylcyclohex-1-en or 2-en-1-yl)-hex-1-en-3-one (according to GC approx. 80%) were hydrogenated with 5 g of 5% rhodium on activated aluminium oxide, corresponding to 0.25 wt. % of rhodium on aluminium oxide, in a stirred autoclave with a gassing stirrer under 130 bar and at a temperature of from 220° C. to 260° C. for 1 hour. Heating up was carried out here in the course of 40 minutes. After filtration and distillation, completely hydrogenated product was obtained. The ratio of trans-/cis-1-(2,2,6-trimethylcyclohexyl)hexan-3-ol was 50:50.

### EXAMPLE 4 (Not According to the Invention)

### Preparation of 1-(2,2,6-trimethylcyclohexyl)-hexan-3-ol in the presence of a Raney nickel catalyst

2,000 g of a mixture of 1-(2,6,6-trimethylcyclohex-1-en or 2-en-1-yl)-hex-1-en-3-one (according to GC approx. 80%) were hydrogenated with 60 g of Raney nickel, corresponding to 3 wt. % of Raney nickel, in a stirred autoclave with a gassing stirrer under a hydrogen pressure of 40 bar and at a reaction temperature of from 280° C. to 300° C. for one hour. Heating up to the reaction temperature was carried out here in the course of 50 minutes. After filtration and distillation, the completely hydrogenated product was obtained. The ratio of trans-:cis-1-(2,2,6-trimethylcyclohexyl)-hexan-3-ol was 1:5 (=16.6:83.3).

### EXAMPLE 5

### Isolation of pure cis-1-(2,2,6-trimethylcyclohexyl)-hexan-3-ols of the formula C

50 g of finely distilled alcohol mixture from Example 4, which contains the two alcohols in a trans/cis ratio of 1:5, were subjected to fine distillation again on a Fischer cracking tube column. 25 g of cis-1-(2,2,6-trimethylcyclohexyl)-hexan-3-ol of the formula C were obtained by this procedure as a colourless oil in a purity of 96% with a residual content of 3% of trans-1-(2,2,6-trimethylcyclohexyl)-hexan-3-ol of the formula B (M1; cf. Example 7). Kp_{1mbar} 138° C.-139° C.

### EXAMPLE 6

40 g of finely distilled alcohol mixture from Example 2 were subjected to fine distillation again on a Fischer cracking tube column. 5 g of trans-1-(2,2,6-trimethylcyclohexyl)-hexan-3-ol of the formula B were obtained by this procedure as a colourless oil in a purity of 97% with a residual content of 2% of cis-1-(2,2,6-trimethylcyclohexyl)-hexan-3-ols of the formula C (M3; cf. Example 7). Kp_{1mbar} 140° C.-141° C.

### EXAMPLE 7

Sensorial comparison study: Mixtures having different contents of the cis and/or trans isomer.

A sensorial evaluation of three samples M1, M2 and M3 was first carried out:

M1 from Example 5

96% cis 1-(2,2,6-trimethylcyclohexyl)hexan-3-ol

M2 from Example 2

49% trans 1-(2,2,6-trimethylcyclohexyl)hexan-3-ol

51% cis 1-(2,2,6-trimethylcyclohexyl)hexan-3-ol.

M3 from Example 6

97% trans 1-(2,2,6-trimethylcyclohexyl)hexan-3-ol

The sensorial evaluation of the three samples M1, M2 and M3 was carried out with a panel of experts of eight persons (four women and four men). The samples were in each case evaluated as a 5 wt. % strength ethanolic solution after substantial evaporation of the ethanol on the odour strip as follows:

M1: 96% cis-1-(2,2,6-trimethylcyclohexyl)hexan-3-ol of the formula C

Diffuse, animal, sweaty with a slightly rancid undertone, weakly ambered with suggestions of irises, the females also found clearly urine-like suggestions.

M2: Mixture of trans- and cis-1-(2,2,6-trimethylcyclohexyl)-hexan-3-ol from Example 2.

Heavy, anaesthetizing, narcotic, intense ambered note, diffuse, very intensive emission with a note reminiscent of natural ambergris, very balanced overall impression.

M3: 97% trans-1-(2,2,6-trimethylcyclohexyl)-hexan-3-ol of the formula B

Animal, faecal overall impression which intensifies as the drying time increases, slightly ambered.

All the samples have an intense after-odour which persists for several weeks, overall the sample M2 prepared according to the invention being distinguished by the most balanced and harmonic overall impression and having the highest odour intensity of the three samples. In the case of samples M1 and M3, the negative odour impressions already described above intensified after several days.

Samples M1, M2 and M3 were employed in the following Example 8.

### EXAMPLE 8

Influence of the mixture ratio of the cis/trans isomers on the sensorial impression of various perfume bases.

The following perfume bases were prepared:

8.1 Perfume base of the flowery-woody type

| | |
|---|---|
| Bergamot oil | 7.5 |
| Linalool | 4.0 |
| Phenylethyl alcohol | 5.0 |
| Benzyl acetate | 2.0 |
| Citronellol | 2.0 |
| Hedione (a) | 10.0 |
| Ysamber - K ® (c) | 5.0 |
| Lyral (b) | 4.0 |
| Hydroxycitronellal | 2.5 |
| Rose oxide (c), 10% in DPG | 2.5 |
| Hexylcinnamaldehyde, alpha | 7.5 |
| Patchouli oil, Indonesian | 4.0 |
| Iso-E-Super (b) | 2.0 |
| Vetiveryl acetate | 2.0 |
| Brahmanol F (c) | 2.0 |
| Benzyl salicylate | 2.0 |
| cis-3-Hexenyl salicylate | 1.0 |
| Cedramber (b) | 1.0 |
| Ambrettolide | 1.0 |
| Indole, 10% in DPG | 0.5 |
| Opoponax extract | 0.5 |
| Oak moss extract, 50% in DPG | 5.0 |
| Lilial | 10.0 |
| Total | 83.0 |

(a) Firmenich
(b) IFF
(c) Symrise

The perfume base of the formula shown has a balanced flowery-woody fragrance character.

8.2 Perfume base of the fern type

| | |
|---|---|
| Bergamot oil | 18.0 |
| Lavandin oil super | 15.0 |
| Lilial | 10.0 |
| Anisaldehyde | 3.0 |
| Coumarin | 5.0 |
| Hexylcinnamaldehyde, alpha | 20.0 |
| Ambrinol epoxide, 10% in DPG | 0.5 |
| Ambroxide (c) | 1.0 |
| Cantryl (c) | 10.0 |
| Peppermint oil | 1.0 |
| Total | 83.5 |

The perfume base of the formula shown has a fresh—herbal fern fragrance.

8.3 Perfume base of the green-flowery type

| | |
|---|---|
| Galbanum oil ED | 0.5 |
| Eugenol | 1.0 |
| Methylionone - gamma | 5.0 |
| cis-3-Hexenyl salicylate | 3.0 |
| Benzyl acetate | 8.0 |
| Brahmanol | 5.0 |
| Hedione (a) | 10.0 |
| Benzyl salicylate | 10.0 |
| Hexylcinnamaldehyde | 10.0 |
| Phenylethyl alcohol | 15.0 |
| Nerol | 10.0 |
| Bourbon geranium oil | 7.5 |
| Ylang-ylang oil | 5.0 |
| Total | 90.0 |

The perfume base of the formula shown has a harmonic, flowery-green fragrance character.

The perfume bases 8.1, 8.2 and 8.3 described were then mixed with the samples M1, M2 and M3 described above (Example 7) and the mixtures were then evaluated sensorially by the panel of experts of eight persons.

(a) Mixtures with base 8.1:

| | (A) | (B) | (C) |
|---|---|---|---|
| Base 8.1 | 83 | 83 | 83 |
| M1 | 17 | — | — |
| M2 | — | 17 | — |
| M3 | — | — | 17 |

The mixtures prepared were evaluated sensorially first in the fresh state and at intervals of 8 and 24 hours to evaluate the after-odours. Mixtures (A) and (C) had an unharmonic, non-rounded, musty overall impression here. In the case of Examples A and C, the initial flowery, woody fragrance character was lost, and the odour intensity thereby shifted into odour impressions with wet powdery suggestions. These odour tonalities reminiscent of wet hide are generally found to be unpleasant. The negative overall impression intensified in particular in the evaluation of the after-odours of mixtures (A) and (C), the sweaty or faecal odour impressions found to be negative in the evaluation of the pure odours intensifying.

On the other hand, mixture (B) not only was distinguished by an increased substantivity, i.e. it remains on the dried odour strip for significantly longer, the flowery—woody odour impression of the original base shifted to a light, slightly diffuse ambergris note with clear suggestions of natural ambergris.

(b) Mixtures with base 8.2

| | (D) | (E) | (F) |
|---|---|---|---|
| Base 8.2 | 83.5 | 83.5 | 83.5 |
| M1 | 16.5 | — | — |
| M2 | — | 16.5 | — |
| M3 | — | | 16.5 |

Mixtures (D) and (F) were distinguished by an overall comparable odour impression. In the area of the top note, on the other hand, a clearly interfering, unharmonically acting slightly sweetish-sourish bad note is found with mixture (D). This makes the mixture seem unbalanced and not so spicy, as is desired with bases of this type. In the case of mixture (F), an intensifying bad note is found in the area of the after-odours. This bad note, which is reminiscent of wet leather, intensified as drying increased. The negative odour impression intensified in additional tests when the weight content of component M3 in the base was increased.

Mixture (E), on the other hand, has harmonic suggestions of bergamot and nutmeg which emphasize the top note and are desired in such notes, the spicy aspects and suggestions of rich wood types intensifying them making the overall impression appear very balanced. It was possible to cover or mask slightly sourish animal odour impression in base 8.2 by admixing M2. M2 not only was distinguished by masking effects, the absorption capacity and adhesion were improved significantly. At the same time, an emphasis of the top note was found in the superficial odour.

(c) Mixtures with base 8.3

The perfume base from Example 9 was mixed with samples M1, M2 and M3 in the stated weight contents, the mixtures were then incorporated separately into a soap formulation and the resulting soap was evaluated sensorially by the panel of experts.

| | (G) | (H) | (J) |
|---|---|---|---|
| Perfume base from Example 9 | 90.0 | 90.0 | 90.0 |
| M1 | 10.0 | — | — |
| M2 | — | 10.0 | — |
| M3 | — | — | 10.0 |

The panel of experts clearly preferred composition (H), this being represented by an overall clearer emission, by more freshness and a very balanced harmonic odour impression.

1. Process for the preparation of 1-(2,2,6-trimethylcyclohexyl)-hexan-3-ol of the formula A 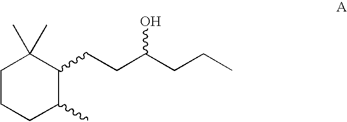
having a content of at least 30 wt. % of the corresponding trans isomer, based on the total amount of 1-(2,2,6-trimethylcyclohexyl)-hexan-3-ol of the formula A, prepared by the following step:
Catalytic hydrogenation of a compound of the formula D 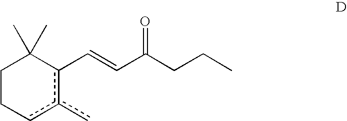
in which the broken lines represent a single double bond which is located in one of the three positions drawn in, in the presence of a rhodium catalyst to give 1-(2,2,6-trimethylcyclohexyl)-hexan-3-ol of the formula A.

2. Process according to claim 1, wherein the hydrogenation is ended only after conversion of at least 98% of the compound of the formula D.

3. Process according to claim 1, wherein the hydrogenation is carried out in a hydrogen pressure range of from 1 to 200 bar, preferably in the range of from 80 to 200 bar, particularly preferably in the range of from 80 to 150 bar.

4. Process according to claim 1, wherein the hydrogenation is carried out in a hydrogen pressure range of from above 100 bar to not more than 200 bar, preferably in the range of from above 100 bar to not more than 150 bar.

5. Process according to claim 1, wherein the catalytic hydrogenation is carried out at a temperature in the range of from 20° C. to 300° C., preferably at a temperature in the range of from 180° C. to 260° C.

6. Process according to claim 1, wherein rhodium is employed in the catalytic hydrogenation in an amount of from 0.0001 to 10 wt. %, preferably in the range of from 0.001 to 0.5 wt. %, based on the amount of compound of the formula D employed.

7. Process according to claim 6, wherein
(a) the 1-(2,2,6-trimethylcyclohexyl)-hexan-3-ol of the formula A prepared is separated off
(i) from the rhodium catalyst and/or
(ii) from other constituents of the product mixture and/or
(b) an isomer of the 1-(2,2,6-trimethylcyclohexyl)-hexan-3-ol of the formula A prepared is separated off from the product mixture or concentrated.

8. Process for the preparation of a perfume composition, comprising the following steps:
Preparation of 1-(2,2,6-trimethylcyclohexyl)-hexan-3-ol by a process according to claim 1,
Optionally isolation of the 1-(2,2,6-trimethylcyclohexyl)-hexan-3-ol from the product mixture and/or purification of the product mixture,
and subsequently
mixing of a sensorially active amount of the 1-(2,2,6-trimethylcyclohexyl)-hexan-3-ol with one or more further odoriferous substances.
